(19) United States
(12) Patent Application Publication
(10) Pub. No.: US 2012/0266906 A1
(43) Pub. Date: Oct. 25, 2012

(54) Composition for Treating Hair, Methods of Preparing the Composition, and Methods of Using the Composition

(75) Inventor: Suzanne Wilson, Windham, NH (US)

(73) Assignee: ORGANIX XTREME, LTD., Windham, NH (US)

(21) Appl. No.: 13/451,513

(22) Filed: Apr. 19, 2012

Related U.S. Application Data
(60) Provisional application No. 61/477,164, filed on Apr. 19, 2011.

(51) Int. Cl. A61K 8/98; A61Q 5/00; A61K 8/97

(52) U.S. Cl. 132/202; 424/70.1; 424/74

(57) ABSTRACT

A composition for treating hair and, in particular, for defrizzing hair, includes blackstrap molasses and honey. Essential oils can be added to the mixture. A method exists for making the composition. The method of making the composition includes heating the composition, possibly to a boil, cooling the composition, adding the essential oils, and mixing the imbibed composition. The composition is used in methods to treat hair. The composition treats hair by application or through heat treatments.

### CROSS-REFERENCE TO RELATED APPLICATIONS

This application claims the benefit of U.S. Provisional Application No. 61/477,164, filed Apr. 19, 2011.

### STATEMENT REGARDING FEDERALLY SPONSORED RESEARCH OR DEVELOPMENT

Not Applicable

### THE NAMES OF PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

The invention relates to compositions for hair treatment, methods of forming compositions, and methods of treating hair.

2. Description of the Related Art

Frizzy hair is a symptom of styled hair that is exposed to humidity and other environmental conditions.

An anti-frizzing treatment for hair is the Brazilian Keratin Treatment. The Brazilian Keratin Treatment is a long-term blow dry for hair that lasts about three months. Pioneered in Brazil, the Brazilian Keratin Treatment is performed on all types of chemically-treated hair (bleached, hi-lights, colored, permed, relaxed or previously straightened). The system aims to eliminate frizz, unruly curls, and waves. The system does not guarantee completely straight hair. It can relax between fifty and eighty percent (50-80%) of the curl depending on texture, if done correctly. Treatments last around 10-12 weeks and repeating the treatment every few months will allow re-treatment of the new growth. The treatment does not allow one to wash the hair for at least 48-72 hours, pick it up with any hair clip, pony tail or locate the hair behind the ears.

Health concerns focus on one chemical used in the process, formaldehyde, which the article notes is a known carcinogen.

### BRIEF SUMMARY OF THE INVENTION

The invention encompasses a composition for treating hair, a method of making the composition, and methods of treating hair with the composition.

Hair treated with the composition shows the following improvements: increased moisture, increased sheen, added body, reduced frizzing, increased protection from heat, increased elasticity, opened hair cuticles, softened hair strands, rejuvenated hair follicles, reduced dry scalp, itching, and dandruff, regulated pH, and enhanced hair growth.

The composition is a mixture of blackstrap molasses and raw honey.

Blackstrap molasses, or simply blackstrap, is the dark, viscous molasses remaining after maximum extraction of sugar from raw sugar cane. This residual product of sugar refining is used in the manufacture of ethyl alcohol for industry and as an ingredient in cattle feed. The term is an Americanism dating to around 1920.

The third boiling of the sugar syrup yields blackstrap molasses. The majority of sucrose from the original juice has been crystallized. Blackstrap molasses contains significant amounts of vitamin B6 and minerals, including calcium, magnesium, potassium, and iron.

A mixture of one volume of blackstrap molasses with four volumes of honey has been found to be particularly effective at treating hair with making the hair brittle or dry.

Essential oils can be added to the composition.

Blackstrap molasses is preferred over refined molasses. Although refined molasses does work as an ingredient in the treatment, blackstrap molasses produced a more “tenacious” product. That is, the resulting composition was more robust and clung better to the hair. The effect of the composition on hair was greater when using blackstrap molasses as compared to refined molasses.

Honey of all types (raw and refined) was an effective ingredient. Using raw honey is preferred by many consumers. Using raw honey supports local beekeepers.

Honey provides a moisturizing effect when added to the hair. Honey acts as a humectant.

The pH of honey is commonly between 3.2 and 4.5. This relatively acidic pH level prevents the growth of many bacteria. The acidic pH helps to buffer the pH of many soaps, which are typically alkaline.

A combination of honey and molasses was found to be preferable over compositions with molasses alone. When molasses was used without honey, the treated hair became dry and brittle.

Honey when used alone was not found to stop frizzing.

When compositions were made with one volume of molasses to one volume of honey, a composition that defrizzes hair is made. However, after repeated weekly treatment with 1:1 compositions, the treated hair begins to dry.

Boiling of the mixture of molasses and honey reduces the amount of yeast in the mixture to levels that are acceptable to manufacturing standards. Without boiling, the mixture can have yeast levels that exceed health standards. Yeast levels were found to be particular high when using unrefined blackstrap molasses and honey.

After boiling, the mixture is allowed to cool to room temperature. The mixture is then allowed to rest for twelve hours.

After cooling, a light gray layer forms on the surface. The layer appears light and caramel colored. With time, the layer becomes softer.

Essential oils are added to improve the sheen of treated hair. Essential oils also provide scent to the composition. The essential oils are added two to three days after the mixture has cooled.

Essential oils are not added to a heated mixture until it has been returned to room temperature. Adding essential oils to a boiling mixture can degrade the essential oils.

The scent of the mixture with oils increases for the first week of rest. Resting the mixture for longer than one week does not increase the scent of the mixture.

The oils are not mixed initially. The oils are allowed to permeate the mixture without disturbing the layer on the top.

The mixture is stirred on the tenth day after boiling.

Excess eucalyptus oil was found to lead to dry hair.

Other features of the invention are set forth in the appended claims.

Although the invention is described herein as embodied in a composition for treating hair, a method of making the composition, and methods of treating hair with the composition, the invention is not limited to the details shown because various modifications and compositional changes may be made without departing from the invention and the equivalents of the claims. However, the construction and method of operation of the invention together with additional objects and advantages thereof will be best understood from the following description of specific embodiments when read in connection with the accompanying drawings.

### BRIEF DESCRIPTION OF THE SEVERAL VIEWS OF THE DRAWING

Not Applicable.

### DETAILED DESCRIPTION OF THE INVENTION

A preferred embodiment of a composition for treating hair includes the following ingredients.

Three (3) cups of raw blackstrap molasses.

Twelve (12) cups of raw honey.

Six drops of rosemary essential oil. A drop is approximately 0.05 mL (50 μL).

Six drops of sage essential oil.

Four (4) drops of eucalyptus oil.

Six (6) drops of jojoba essential oil.

Six (6) drops of clove bud essential oil.

A first preferred embodiment of a method of making the composition includes the following sequence of steps.

Three cups of blackstrap molasses are mixed with twelve cups of raw honey to create a mixture. The mixture is heated to a boil. The mixture is then cooled to room temperature. The room-temperature mixture is stirred approximately twenty times. The essential oils are added to the room-temperature mixture. The order of adding the essential oils is not significant. The following essential mixtures are admixed: six (6) drops of rosemary essential oil, six (6) drops of sage essential oil, four (4) drops of eucalyptus essential oil, six (6) drops of jojoba essential oil, and six (6) drops of clove bud essential oil. The mixture including the essential oils is mixed, approximately two-hundred turns is adequate.

A second preferred embodiment of a method of making the composition includes the following sequence of steps.

Three cups of blackstrap molasses are mixed with twelve cups of raw honey to create a mixture. The mixture is heated to a boil. The mixture is then cooled to room temperature. The room-temperature mixture is allowed to rest between two and three days. The essential oils are added to the room-temperature mixture. The order of adding the essential oils is not significant. The following essential mixtures are admixed: six (6) drops of rosemary essential oil, six (6) drops of sage essential oil, four (4) drops of eucalyptus essential oil, six (6) drops of jojoba essential oil, and six (6) drops of clove bud essential oil. After 7-10 days, the mixture including the essential oils is mixed, approximately two-hundred turns is adequate.

Three preferred embodiments of methods of using the composition include a deep treatment with heat, a deep treatment without heat, and a daily treatment.

The first method of treatment is for a deep treatment when a heating cap or commercial hair dryer is available. The first step of the method is shampooing the hair. The second step is towel drying the hair. The third step is applying the composition to the hair. Typically two (2) tablespoons are sufficient. Enough composition is used to cover the shaft of the hair. Next, a cap is placed over the hair. Next, a hair dryer or heating cap is applied to the hair for approximately thirty minutes. The cap is then removed. Next, the composition is rinsed from the hair with warm water. Next, conditioner is applied to the hair. In the final step, the hair is styled and or blown dry.

The method results in hair with improved shine and reduced fizzing. Treated hair has increased body and strength (i.e. resists breaking). The treatment shortens drying time and improves texture. Treated hair requires less hair products for styling than untreated hair.

The first method of applying the treatment is preferably performed weekly.

The second method of treating hair with the composition is for users without access to a heating/steam cap or dryer.

The first step of the second method is to shampoo the hair. The second step is towel dry the hair. The next step is to apply the composition with at least enough of the composition to cover the shaft of the hair. Next, a cap is placed over the hair. The hair is treated between ten (10) minutes and one (1) hour. In the next step, the treatment is rinsed from the hair. Then, the hair is conditioned. Lastly, the hair is styled.

Preferably, the second method of treatment is performed once per week.

The third preferred method of using the composition for treating the hair is the “easy” method.

In the third method, the first step is shampooing the hair. Next, the composition is applied to the hair. Sufficient composition is applied to coat the shaft of the hair. The composition is left in the hair for up to thirty (30) minutes. The composition is not rinsed from the hair. Conditioner is added to the hair. Next, the composition and conditioner are rinsed from the hair. Lastly, the hair is dried and/or styled.

What is claimed is:

1. A composition for treating hair, comprising:
molasses; and
honey.

2. The composition according to claim 1, wherein said molasses is blackstrap molasses.

3. The composition according to claim 1, wherein said honey is raw honey.

4. The composition according to claim 1, further comprising an essential oil.

5. The composition according to claim 1, wherein one volume of said molasses is mixed with one to ten volumes of honey.

6. The composition according to claim 1, wherein one volume of said molasses is mixed with four volumes of honey.

7. The composition for treating hair according to claim 1, further comprising:
six drops of rosemary essential oil;
six drops of sage essential oil;
four drops of eucalyptus oil;
six drops of jojoba essential oil; and
six drops of clove bud essential oil;
said molasses being raw blackstrap molasses;
said honey being raw honey; and
said raw blackstrap molasses being mixed at a ratio of three cups of said raw blackstrap molasses to twelve cups of said raw honey.

8. A method of making a composition for treating hair, which comprises:
providing molasses;
admixing honey to create a mixture; and
heating the mixture to a boil.

9. The method according to claim 8, which further comprises cooling the mixture to room temperature.

10. The method according to claim 9, which further comprises adding an essential oil to the mixture after cooling.

11. The method according to claim 10, wherein said adding step is performed at least two days after the cooling step.

12. The method according to claim 10, which further comprises mixing said essential oil with stirring at least seven days after the cooling step.

13. The method according to claim 8, which further comprises applying the mixture to hair.

14. A method of treating hair, which comprises applying a composition to hair, the composition including molasses and honey.

15. The method according to claim 14, which further comprises heating the hair before rinsing the composition from the hair.

16. The method according to claim 14, which further comprises shampooing the hair before applying the composition.

17. The method according to claim 14, which further comprises:
treating the hair with the composition for a time between ten minutes and one hour;

18. The method according to claim 14, which further comprises:
treating the hair with the composition for up to thirty minutes;
rinsing the composition from the hair;
applying conditioner to the hair;
rinsing the hair; and
styling the hair.
